(19) United States
(12) Patent Application Publication
(10) Pub. No.: US 2007/0105928 A1
(43) Pub. Date: May 10, 2007

(54) 6-Pentafluorophenyl-triazolopyrimidines, method for their production and their use for combating pathogenic fungi, in addition to agents containing said substances

(76) Inventors: Jordi Tormo I Blasco, Laudenbach (DE); Carsten Blettner, Mannheim (DE); Bernd Muller, Frankenthal (DE); Markus Gewehr, Kastellaun (DE); Wassilios Grammenos, Ludwigshafen (DE); Thomas Grote, Wachenheim (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Maria Scherer, Godramstein (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 10/582,938

(22) PCT Filed: Dec. 14, 2004
(86) PCT No.: PCT/EP04/14210

§ 371 Date: Jun. 15, 2006

(30) Foreign Application Priority Data
Dec. 17, 2003 (DE) 103594452.3

(51) Int. Cl. A61K 31/41

(52) U.S. Cl. 514/384

(57) ABSTRACT

6-Pentafluorophenyltriazolopyrimidines of the formula I 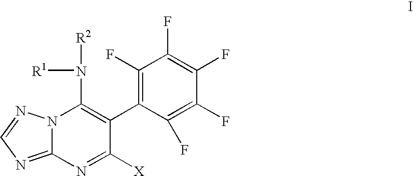 in which the substituents are as defined below:
R¹ is alkyl, haloalkyl, cycloalkyl, halocycloalkyl, alkenyl, haloalkenyl, cycloalkenyl, halocycloalkenyl, alkinyl, haloalkinyl or phenyl, naphthyl, or a five- or six-membered saturated, partially unsaturated or aromatic heterocycle which contains one to four heteroatoms from the group consisting of O, N and S,
R² is hydrogen or one of the groups mentioned under R¹,
   R¹ and R² together with the nitrogen atom to which they are attached may also form a five- or six-membered heterocyclyl or heteroaryl which is attached via N and may contain one to three further heteroatoms from the group consisting of O, N and S as ring members;
   R¹ and/or R² may be substituted as defined in the description; X is cyano, alkyl, alkoxy, alkenyloxy, haloalkoxy or haloalkenyloxy, processes for preparing these compounds, compositions comprising them and their use for controlling phytopathogenic harmful fungi.

The present invention relates to 6-pentafluorophenyltriazolopyrimidines of the formula I 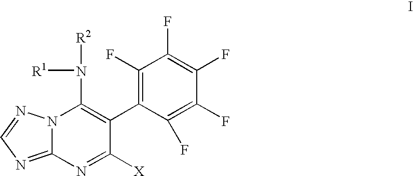
in which the substituents are as defined below:
R¹ is C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl or a five- or six-membered saturated, partially unsaturated or aromatic heterocycle which contains one to four heteroatoms from the group consisting of O, N and S,
R² is hydrogen or one of the groups mentioned under R¹,
   R¹ and R² together with the nitrogen atom to which they are attached may also form a five- or six-membered heterocyclyl or heteroaryl which is attached via N and may contain one to three further heteroatoms from the group consisting of O, N and S as ring members and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy;
   R¹ and/or R² may carry one to four identical or different groups R^{a}:
      R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₈-alkenyl, C₂-C₈-haloalkenyl, C₂-C₆-alkenyloxy, C₂-C₈-alkynyl, C₂-C₈-haloalkynyl, C₃-C₆-alkynyloxy, oxy-C₁-C₃-alkyleneoxy, C₃-C₈-cycloalkenyl, phenyl, naphthyl, a five- or six-membered saturated, partially unsaturated or aromatic heterocycle which contains one to four heteroatoms from the group consisting of O, N and S, where these aliphatic, alicyclic or aromatic groups for their part may be partially or fully halogenated;
X is cyano, C₁-C₄-alkyl, C₁-C₄-alkoxy, C₃-C₄-alkenyloxy, C₁-C₂-haloalkoxy or C₃-C₄-haloalkenyloxy.

Moreover, the invention relates to a process for preparing these compounds, to compositions comprising them and to their use for controlling phytopathogenic harmful fungi.

5-Halo-6-pentafluorotriazolopyrimidines are known in a general manner from EP-A 834 513. 5-Cyano- and 5-alkoxytriazolopyrimidines are disclosed in WO 02/083677. Triazolopyrimidines having optically active amino substituents in the 7-position are proposed in a general manner in WO 02/38565.

The compounds described in the publications mentioned above are suitable for controlling harmful fungi.

However, their action is not always entirely satisfactory. Using this as a basis, it is an object of the present invention to provide compounds having improved activity and/or a broader activity spectrum.

Accordingly, we have found the compounds defined at the outset. Moreover, we have found a process for their preparation, compositions comprising them and methods for controlling harmful fungi using the compounds I.

The compounds according to the invention differ from those described in the abovementioned publication by the substitution in the 5-position of the triazolopyrimidine skeleton.

Compared to the known compounds, the compounds of the formula I have increased activity and/or a broader activity spectrum against harmful fungi.

The compounds according to the invention can be obtained by different routes. Advantageously, they are obtained from the 5-halo-6-pentafluorophenyltriazolopyrimidines of the formula II known from EP-A 834 513 by reaction with compounds M-X (formula II). Depending on the meaning of the group X to be introduced, the compounds III are inorganic cyanides or alkoxides. The reaction is advantageously carried out in the presence of an inert solvent. The cation M in the formula III is of little importance; for practical reasons, ammonium, tetraalkylammonium or alkali metal or alkaline earth metal salts are usually preferred.

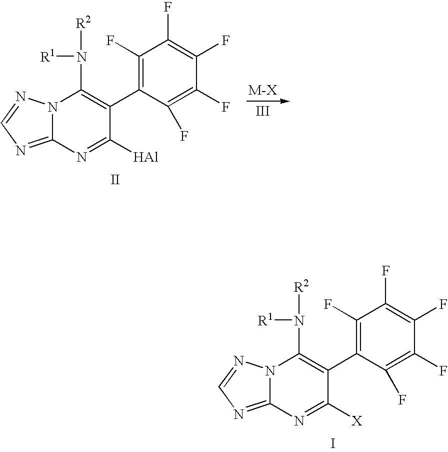

The reaction temperature is usually from 0 to 120° C., preferably from 10 to 40° C. [cf. J. Heterocycl. Chem., Vol. 12, pp. 861-863 (1975)].

If R² is hydrogen, a removable protective group is advantageously introduced prior to the reaction with III [cf. Greene, Protective Groups in Organic Chemistry, J. Wiley & Sons, (1981)].

Suitable solvents comprise ethers, such as dioxane, diethyl ether and, preferably, tetrahydrofuran, alcohols, such as methanol or ethanol, halogenated hydrocarbons, such as dichloromethane, and aromatic hydrocarbons, such as toluene or acetonitrile.

Compounds of the formula I in which X is C₁-C₄-alkyl can advantageously be obtained by the following synthesis route:

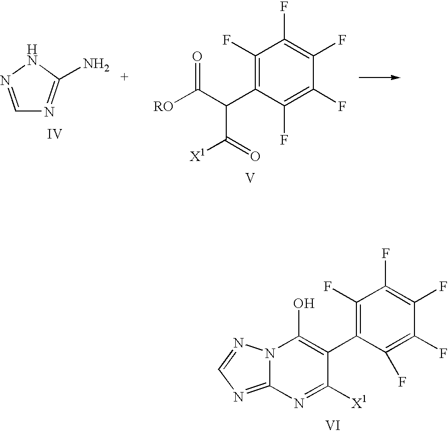

Starting with 2-aminotriazole IV and ketoesters V where R is C₁-C₄-alkyl, the 5-alkyl-7-hydroxy-6-phenyltriazolopyrimidines VI are obtained. In the formulae V and VI, X¹ is C₁-C₄-alkyl. Using the easily obtainable 2-phenylacetoacetic esters (V where X¹═CH₃), the 5-methyl-7-hydroxy-6-phenyltriazolopyrimidines, which are a preferred subject matter of the invention, are obtained [cf. Chem. Pharm. Bull., 9, 801, (1961)]. 2-Aminotriazole IV is commercially available. The starting materials V are advantageously prepared under the conditions described in EP-A 10 02 788.

The resulting 5-alkyl-7-hydroxy-6-phenyltriazolopyrimidines are converted with halogenating agents [HAL] under the conditions known from WO-A 94/20501 into the halopyrimidines of the formula VII in which Hal is a halogen atom, preferably a bromine or a chlorine atom, in particular a chlorine atom. The halogenating agent [HAL] used is advantageously a chlorinating or brominating agent, such as phosphorus oxybromide, phosphorus oxychloride, thionyl chloride, thionyl bromide or sulfuryl chloride.

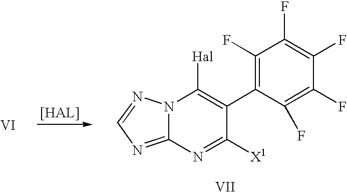

This reaction is usually carried out at from 0° C. to 150° C., preferably at from 80° C. to 125° C. [cf. EP-A 770 615].

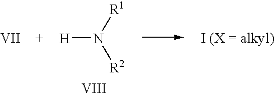

The reaction of VII with amines VIII where R¹ and R² are as defined in formula I is advantageously carried out at from 0° C. to 70° C., preferably from 10° C. to 35° C., preferably in the presence of an inert solvent, such as an ether, for example dioxane, diethyl ether or, in particular, tetrahydrofuran, a halogenated hydrocarbon, such as dichloromethane, or an aromatic hydrocarbon, such as, for example. toluene [cf. WO-A 98/46608].

The use of a base, such as a tertiary amine, for example triethylamine, or an inorganic amine, such as potassium carbonate, is preferred; it is also possible for excess amine of the formula VIII to serve as base.

Alternatively, compounds of the formula I, in which X is C₁-C₄-alkyl can also be prepared from compounds I in which X is halogen, in particular chlorine, and malonates of the formula IX. In the formula IX, X″ is hydrogen or C₁-C₃-alkyl and R is C₁-C₄-alkyl. They are converted into compounds of the formula X and decarboxylated to give the compounds I [cf. U.S. Pat. No. 5,994,360].

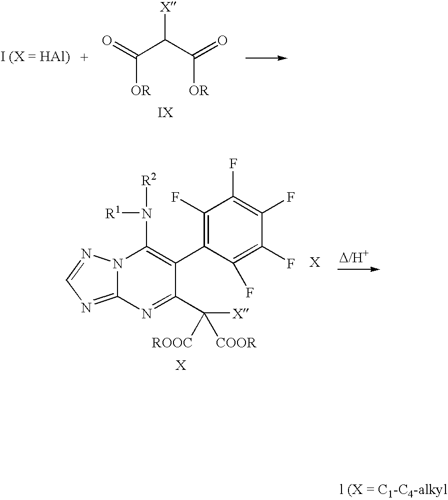

The malonates IX are known from the literature [J. Am. Chem. Soc., 64, 2714 (1942); J. Org. Chem., 39, 2172 (1974); Helv. Chim. Acta, 61, 1565 (1978)], or they can be prepared in accordance with the literature cited.

The subsequent hydrolysis of the ester X is carried out under generally customary conditions; depending on the various structural elements, alkaline or acidic hydrolysis of the compounds X may be advantageous. Under the conditions of the ester hydrolysis, there may be complete or partial decarboxylation, giving I.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-touenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; particularly preferably, the reaction is carried out in hydrochloric acid or acetic acid. It is also possible to use mixtures of the solvents mentioned.

Compounds of the formula I in which X is C₁-C₄-alkyl can also be prepared by coupling 5-halotriazolopyrimidines of the formula I in which X is halogen with organometallic reagents of the formula XI. In one embodiment of this process, the reaction is carried out with transition metal catalysis, such as Ni or Pd catalysis.

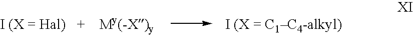

In formula XI, M is a metal ion of valency y, such as, for example, B, Zn or Sn, and X″ is C₁-C₃-alkyl. This reaction can be carried out, for example, analogously to the following methods: J. Chem. Soc. Perkin Trans. 1, 1187 (1994), ibid. 1, 2345 (1996); WO-A 99/41255; Aust. J. Chem., vol. 43, 733 (1990); J. Org. Chem., vol. 43, 358 (1978); J. Chem. Soc. Chem. Commun. 866 (1979); Tetrahedron Lett., vol. 34, 8267 (1993); ibid. vol. 33, 413 (1992).

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

In the definitions of the symbols given in the formulae above, collective terms were used which are generally representative of the following substituents:

halogen: fluorine, chlorine, bromine and iodine;

alkyl: saturated straight-chain or branched hydrocarbon radicals having 1 to 4, 6 or 8 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4, 6 or 8 carbon atoms (as mentioned above), where in these groups some or all of the hydrogen atoms may be replaced by halogen atoms as mentioned above; in particular, C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: unsaturated straight-chain or branched hydrocarbon radicals having 2 to 4, 6, 8 or 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: unsaturated straight-chain or branched hydrocarbon radicals having 2 to 8 carbon atoms and one or two double bonds in any position (as mentioned above), where in these groups some or all of the hydrogen atoms may be replaced by halogen atoms as mentioned above, in particular by fluorine, chlorine and bromine;

alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;

five- or six-membered saturated, partially unsaturated or aromatic heterocycle which comprises one to four heteroatoms from the group consisting of O, N and S:

5 or 6-membered heterocyclyl which comprises one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;
   alkylene: saturated straight-chain or branched hydrocarbon radicals having 1 to 4 or 6 carbon atoms, which radicals are attached to the skeleton via a double bond, for example ═CH₂, ═CH—CH₃, ═CH—CH₂—CH₃;
   oxyalkyleneoxy: divalent unbranched chains of 1 to 3 CH₂ groups, where both valences are attached to the skeleton via an oxygen atom, for example OCH₂O, OCH₂CH₂O and OCH₂CH₂CH₂O.

The scope of the present invention includes the (R)- and (S)-isomers and the racemates of compounds of the formula I having chiral centers.

With a view to the intended use of the triazolopyrimidines of the formula I, particular preference is given to the following meanings of the substituents, in each case on their own or in combination:

Preference is given to compounds I in which R¹ is C₁-C₄-alkyl, C₂-C₆-alkenyl or C₁-C₈-haloalkyl.

Preference is given compounds I in which R¹ is a group A: 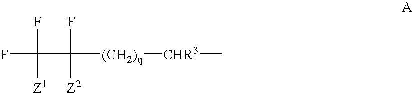
in which
Z¹ is hydrogen, fluorine or C₁-C₆-fluoroalkyl,
Z² is hydrogen or fluorine, or
   Z¹ and Z² together form a double bond;
q is 0 or 1; and
R³ is hydrogen or methyl.

Moreover, preference is given to compounds I in which R¹ is C₃-C₆-cycloalkyl which may be substituted by C₁-C₄-alkyl.

Particular preference is given to compounds I in which R² is hydrogen.

Preference is likewise given to compounds I in which R² is methyl or ethyl.

If R¹ and/or R² comprise haloalkyl or haloalkenyl groups having a center of chirality, the (S)-isomers are preferred for these groups. In the case of halogen-free alkyl or alkenyl groups having a center of chirality in R¹ or R² preference is given to the (R)-configured isomers.

A preferred embodiment of the invention relates to compounds of the formula I.1: 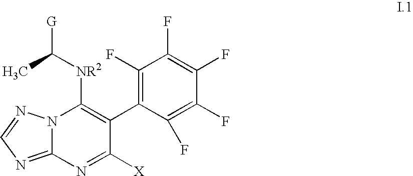
in which
G is C₂-C₆-alkyl, in particular ethyl, n- and isopropyl, n-, sec-, tert-butyl, and C₁-C₄-alkoxymethyl, in particular ethoxymethyl, or C₃-C₆-cycloalkyl, in particular cyclopentyl or cyclohexyl;
R² is hydrogen or methyl; and
X is as defined in formula I, in particular cyano, methoxy or ethoxy.

A further preferred embodiment of the invention relates to compounds of the formula I.2. 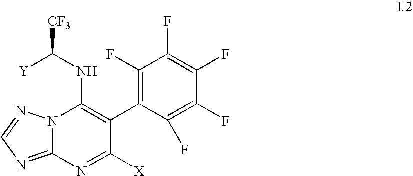
in which Y is hydrogen or C₁-C₄-alkyl, in particular methyl and ethyl, and X is as defined in formula I, in particular represents cyano, methoxy or ethoxy.

A further preferred embodiment of the invention relates to compounds in which R¹ and R² together with the nitrogen atom to which they are attached form a five- or six-membered heterocyclyl or heteroaryl which is attached via N and may contain a further heteroatom from the group consisting of O, N and S as ring member and/or may carry one or more substituents form the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkyleneoxy. These compounds correspond in particular to formula I.3, 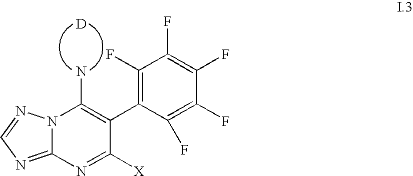
in which
D together with the nitrogen atom forms a five- or six-membered heterocyclyl or heteroaryl which is attached via N and may contain a further heteroatom from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy; and
X is as defined in formula I, in particular cyano, methoxy or ethoxy.

Preference is furthermore given to compounds I in which R¹ and R² together with the nitrogen atom to which they are attached form a piperidinyl, morpholinyl or thiomorpholinyl ring, in particular a piperidinyl ring, which is optionally substituted by one to three groups halogen, C₁-C₄-alkyl or C₁-C₄-haloalkyl. Particular preference is given to the compounds in which R¹ and R² together with the nitrogen atom to which they are attached form a 4-methylpiperidine ring.

The invention furthermore preferably provides compounds I in which R¹ and R² together with the nitrogen atom to which they are attached form a pyrazole ring which is optionally substituted by one or two groups halogen, C₁-C₄-alkyl or C₁-C₄-haloalkyl, in particular by 3,5-dimethyl or 3,5-di(trifluoromethyl).

In addition, particular preference is also given to compounds of the formula I in which R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl; R² is hydrogen or methyl; or R¹ and R² together are —(CH₂)₂CH(CH₃)(CH₂)₂—, —(CH₂)₂CH(CF₃)(CH₂)₂— or —(CH₂)₂O(CH₂)₂—.

Particular preference is furthermore given to compounds I in which X is cyano, methoxy or ethoxy, in particular cyano or methoxy.

In a further preferred embodiment of the compounds I X is methyl.

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

Table 1

Compounds of the formula I in which X is cyano and the combination of R¹ and R² corresponds for each compound to one row of table A.

Table 2

Compounds of the formula I in which X is methoxy and the combination of R¹ and R² corresponds for each compound to one row of table A.

Table 3

Compounds of the formula I in which X is ethoxy and the combination of R¹ and R² corresponds for each compound to one row of table A.

Table 4

Compounds of the formula I in which X is methyl and the combination of R¹ and R² corresponds for each compound to one row of table A.

TABLE A

| No. | R¹ | R² |
|---|---|---|
| A-1 | CH₃ | H |
| A-2 | CH₃ | CH₃ |
| A-3 | CH₂CH₃ | H |
| A-4 | CH₂CH₃ | CH₃ |
| A-5 | CH₂CH₃ | CH₂CH₃ |
| A-6 | CH₂CF₃ | H |
| A-7 | CH₂CF₃ | CH₃ |
| A-8 | CH₂CF₃ | CH₂CH₃ |
| A-9 | CH₂CCl₃ | H |
| A-10 | CH₂CCl₃ | CH₃ |
| A-11 | CH₂CCl₃ | CH₂CH₃ |
| A-12 | CH₂CH₂CH₃ | H |
| A-13 | CH₂CH₂CH₃ | CH₃ |
| A-14 | CH₂CH₂CH₃ | CH₂CH₃ |
| A-15 | CH₂CH₂CH₃ | CH₂CH₂CH₃ |
| A-16 | CH(CH₃)₂ | H |
| A-17 | CH(CH₃)₂ | CH₃ |
| A-18 | CH(CH₃)₂ | CH₂CH₃ |
| A-19 | CH₂CH₂CH₂CH₃ | H |
| A-20 | CH₂CH₂CH₂CH₃ | CH₃ |
| A-21 | CH₂CH₂CH₂CH₃ | CH₂CH₃ |
| A-22 | CH₂CH₂CH₂CH₃ | CH₂CH₂CH₃ |
| A-23 | CH₂CH₂CH₂CH₃ | CH₂CH₂CH₂CH₃ |
| A-24 | (±)CH(CH₃)—CH₂CH₃ | H |
| A-25 | (±)CH(CH₃)—CH₂CH₃ | CH₃ |
| A-26 | (±)CH(CH₃)—CH₂CH₃ | CH₂CH₃ |
| A-27 | (S)CH(CH₃)—CH₂CH₃ | H |
| A-28 | (S)CH(CH₃)—CH₂CH₃ | CH₃ |
| A-29 | (S)CH(CH₃)—CH₂CH₃ | CH₂CH₃ |
| A-30 | (R)CH(CH₃)—CH₂CH₃ | H |
| A-31 | (R)CH(CH₃)—CH₂CH₃ | CH₃ |
| A-32 | (R)CH(CH₃)—CH₂CH₃ | CH₂CH₃ |
| A-33 | (±)CH(CH₃)—CH(CH₃)₂ | H |
| A-34 | (±)CH(CH₃)—CH(CH₃)₂ | CH₃ |
| A-35 | (±)CH(CH₃)—CH(CH₃)₂ | CH₂CH₃ |
| A-36 | (S)CH(CH₃)—CH(CH₃)₂ | H |
| A-37 | (S)CH(CH₃)—CH(CH₃)₂ | CH₃ |
| A-38 | (S)CH(CH₃)—CH(CH₃)₂ | CH₂CH₃ |
| A-39 | (R)CH(CH₃)—CH(CH₃)₂ | H |
| A-40 | (R)CH(CH₃)—CH(CH₃)₂ | CH₃ |
| A-41 | (R)CH(CH₃)—CH(CH₃)₂ | CH₂CH₃ |
| A-42 | (±)CH(CH₃)—C(CH₃)₃ | H |
| A-43 | (±)CH(CH₃)—C(CH₃)₃ | CH₃ |
| A-44 | (±)CH(CH₃)—C(CH₃)₃ | CH₂CH₃ |
| A-45 | (S)CH(CH₃)—C(CH₃)₃ | H |
| A-46 | (S)CH(CH₃)—C(CH₃)₃ | CH₃ |
| A-47 | (S)CH(CH₃)—C(CH₃)₃ | CH₂CH₃ |
| A-48 | (R)CH(CH₃)—C(CH₃)₃ | H |
| A-49 | (R)CH(CH₃)—C(CH₃)₃ | CH₃ |
| A-50 | (R)CH(CH₃)—C(CH₃)₃ | CH₂CH₃ |
| A-51 | (±)CH(CH₃)—CF₃ | H |
| A-52 | (±)CH(CH₃)—CF₃ | CH₃ |
| A-53 | (±)CH(CH₃)—CF₃ | CH₂CH₃ |
| A-54 | (S)CH(CH₃)—CF₃ | H |
| A-55 | (S)CH(CH₃)—CF₃ | CH₃ |
| A-56 | (S)CH(CH₃)—CF₃ | CH₂CH₃ |
| A-57 | (R)CH(CH₃)—CF₃ | H |
| A-58 | (R)CH(CH₃)—CF₃ | CH₃ |
| A-59 | (R)CH(CH₃)—CF₃ | CH₂CH₃ |
| A-60 | (±)CH(CH₃)—CCl₃ | H |
| A-61 | (±)CH(CH₃)—CCl₃ | CH₃ |
| A-62 | (±)CH(CH₃)—CCl₃ | CH₂CH₃ |
| A-63 | (S)CH(CH₃)—CCl₃ | H |
| A-64 | (S)CH(CH₃)—CCl₃ | CH₃ |
| A-65 | (S)CH(CH₃)—CCl₃ | CH₂CH₃ |
| A-66 | (R)CH(CH₃)—CCl₃ | H |
| A-67 | (R)CH(CH₃)—CCl₃ | CH₃ |
| A-68 | (R)CH(CH₃)—CCl₃ | CH₂CH₃ |
| A-69 | CH₂CF₂CF₃ | H |
| A-70 | CH₂CF₂CF₃ | CH₃ |
| A-71 | CH₂CF₂CF₃ | CH₂CH₃ |
| A-72 | CH₂(CF₂)₂CF₃ | H |
| A-73 | CH₂(CF₂)₂CF₃ | CH₃ |
| A-74 | CH₂(CF₂)₂CF₃ | CH₂CH₃ |
| A-75 | CH₂C(CH₃)═CH₂ | H |
| A-76 | CH₂C(CH₃)═CH₂ | CH₃ |
| A-77 | CH₂C(CH₃)═CH₂ | CH₂CH₃ |
| A-78 | CH₂CH═CH₂ | H |
| A-79 | CH₂CH═CH₂ | CH₃ |
| A-80 | CH₂CH═CH₂ | CH₂CH₃ |
| A-81 | CH₂—C≡CH | H |
| A-82 | CH₂—C≡CH | CH₃ |
| A-83 | CH₂—C≡CH | CH₂CH₃ |
| A-84 | cyclopentyl | H |
| A-85 | cyclopentyl | CH₃ |
| A-86 | cyclopentyl | CH₂CH₃ |
| A-87 | cyclohexyl | H |
| A-88 | cyclohexyl | CH₃ |
| A-89 | cyclohexyl | CH₂CH₃ |
| A-90 | CH₂—C₆H₅ | H |
| A-91 | CH₂—C₆H₅ | CH₃ |
| A-92 | CH₂—C₆H₅ | CH₂CH₃ |

| | |
|---|---|
| A-93 | —(CH₂)₂CH═CHCH₂— |
| A-94 | —(CH₂)₂C(CH₃)═CHCH₂— |
| A-95 | —(CH₂)₂CH(CH₃)(CH₂)₂— |
| A-96 | —(CH₂)₃CHFCH₂— |
| A-97 | —(CH₂)₂CHF(CH₂)₂— |
| A-98 | —CH₂CHF(CH₂)₃— |
| A-99 | —(CH₂)₂CH(CF₃)(CH₂)₂— |
| A-100 | —(CH₂)₂O(CH₂)₂— |
| A-101 | —(CH₂)₂S(CH₂)₂— |
| A-102 | —(CH₂)₅— |
| A-103 | —(CH₂)₄— |
| A-104 | —CH₂CH═CHCH₂— |
| A-105 | —CH(CH₃)(CH₂)₃— |
| A-106 | —CH₂CH(CH₃)(CH₂)₂— |
| A-107 | —CH(CH₃)—(CH₂)₂—CH(CH₃)— |
| A-108 | —CH(CH₃)—(CH₂)₄— |
| A-109 | —CH₂—CH(CH₃)—(CH₂)₃— |
| A-110 | —(CH₂)—CH(CH₃)—CH₂—CH(CH₃)—CH₂— |
| A-111 | —CH(CH₂CH₃)—(CH₂)₄— |
| A-112 | —(CH₂)₂—CHOH—(CH₂)₂— |
| A-113 | —(CH₂)—CH═CH—(CH₂)₂— |
| A-114 | —(CH₂)₆— |
| A-115 | —CH(CH₃)—(CH₂)₅— |
| A-116 | —(CH₂)₂—N(CH₃)—(CH₂)₂— |
| A-117 | —N═CH—CH═CH— |
| A-118 | —N═C(CH₃)—CH═C(CH₃)— |
| A-119 | —N═C(CF₃)—CH═C(CF₃)— |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, corn, grass, bananas, cotton, soybean, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Altemaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g, preferably 5 to 100 g, per 100 kilogram of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

A Water-Soluble Concentrates (SL)

10 parts by weight of a compound according to the invention are dissolved in water or in a water-soluble solvent. As an alternative, wetters or other auxiliaries are added. The active compound dissolves upon dilution with water.

B Dispersible Concentrates (DC)

20 parts by weight of a compound according to the invention are dissolved in cyclohexanone with addition of a dispersant, for example polyvinylpyrrolidone. Dilution with water gives a dispersion.

C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives an emulsion.

D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

E Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.

F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

G Water-Dispersible Powders and Water-Soluble Powders (WP, SP)

75 parts by weight of a compound according to the invention are ground in a rotor-stator mill with addition of dispersants, wetters and silica gel. Dilution with water gives a stable dispersion or solution of the active compound.

2. Products to be Applied Undiluted

H Dustable Powders (DP)

5 parts by weight of a compound according to the invention are ground finely and mixed intimately with 95% of finely divided kaolin. This gives a dustable product.

I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

J ULV Solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier.

Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, enilconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the synthesis examples below were used to prepare further compounds I by appropriate modification of the starting compounds. The compounds thus obtained are listed in the tables below, together with physical data.

### Example 1

### Preparation of 5-cyano-6-pentafluorophenyl-7-diethylamino-1,2,4-triazolo[1,5a]pyrimidine

At 20-25° C., 3.4 g (8.6 mmol) of 5-chloro-6-pentafluorophenyl-7-diethylamino-1,2,4-triazolo[1,5a]pyrimidine and 3.2 g (20 mmol) of tetraethylammonium cyanide were stirred in 30 ml of dimethylformamide for about 45 min. The reaction mixture was diluted with water and the aqueous phase was then extracted with methyl t-butyl ether and the combined organic phases were dried and freed from the solvent. The residue gave, after chromatography on silica gel (cyclohexane/ethyl acetate mixtures), 0.7 g of the title compound as colorless crystalline material of m.p. 153-158° C.

¹H-NMR (CDCl₃, δ in ppm): 8.55 (s, 1H); 3.55 (q, 4H); 1.2 (t, 6H)

### Example 2

### Preparation of 5-methyl-6-(pentafluorophenyl)-7-(4-methylpiperidinyl)-1,2,4-triazolo[1,5a]pyrimidine

### Step 2a: Ethyl 2-pentafluorophenylacetoacetate (Formula V)

A solution of 14.1 g (0.102 mol) of potassium carbonate, 13 g (0.1 mol) of ethyl acetoacetate and 20 g (0.108 mol) of hexafluorobenzene in 100 ml of dimethylformamide (DMF) was stirred at 60° C. for about 5 hours. The reaction mixture was then cooled to 20-25° C. and poured into a mixture of 30 ml of concentrated hydrochloric acid in 700 ml of water. The aqueous phase was extracted with methyl t-butyl ether (MTBE) and the combined organic phases were then dried and freed from the solvent. Chromatography on silica gel (cyclohexane) gave 7.3 g of the title compound as a pale oil.

¹H-NMR (CDCl₃, δ in ppm): 13.4 (s, 1H); 4.2 (q, 2H); 1.9 (s, 3H); 1.2 (t, 3H).

### Step 2b: 5-Methyl-6-(pentafluorophenyl)-7-hydroxy-1,2,4-triazolo[1,5a]pyrimidine (Formula VI)

A solution of 20 g (67 mmol) of ethyl 2-pentafluorophenylacetoacetate (from ex. 2a) and 5.7 g (67 mmol) of aminotriazole in 67 ml of propionic acid was heated under reflux for about 15 hours. The mixture was then cooled to 20-25° C., the volatile components were distilled off and the residue was digested with water. The resulting precipitate was filtered off and taken up in ethyl acetate. The organic phase was washed with dilute hydrochloric acid and dried, and the solvent was removed. The residue crystallized and was digested with hexane. This gave 8 g of a colorless solid comprising the title compound 5-methyl-6-(pentafluorophenyl)-7-hydroxy-1,2,4-triazolo[1,5a]pyrimidine and the isomeric 7-methyl-6-(pentafluorophenyl)-5-hydroxy-1,2,4-triazolo[1,5a]pyrimidine in a ratio of 1:2 (according to ¹H-NMR).

¹H-NMR (DMSO-d₆, δ in ppm):

5-methyl,7-hydroxy isomer: 8.4 (s, 1H); 2.25 (s, 3H).

5-hydroxy,7-methyl isomer: 8.25 (s, 1H); 2.5 (s, 3H).

### Step 2c: 5-Methyl-6-(pentafluorophenyl)-7-chloro-1,2,4-triazolo-[1,5a]pyrimidine (Formula VII)

7 g of the product mixture from example 2b and three drops of DMF in 24 ml of phosphorus oxychloride were heated under reflux for about 4 hours. After cooling, volatile components were removed from the reaction mixture. The residue was taken up in 70 ml of methylene chloride and 30 ml of water, resulting in the methylene chloride starting to boil. After phase separation, the aqueous phase was extracted with methylene chloride. The combined organic phases were dried and freed from the solvent. Chromatography on silica gel (MTBE) gave 4.7 g of a white solid comprising the title compound 5-methyl-6-(pentafluorophenyl)-7-chloro-1,2,4-triazolo-[1,5a]pyrimidine and the isomeric 7-methyl-6-(pentafluorophenyl)-5-chloro-1,2,4-triazolo[1,5a]pyrimidine in a ratio of 4:1 (according to ¹H-NMR).

¹H-NMR (DMSO-d₆, δ in ppm):

5-methyl,7-chloro isomer: 8.9 (s, 1H); 2.75 (s, 3H).

5-chloro,7-methyl isomer: 8.85 (s, 1H); 2.5 (s, 3H).

### Step 2d: 5-Methyl-6-(pentafluorophenyl)-7-(4-methylpiperidinyl)-1,2,4-triazolo-[1,5a]pyrimidine (Formula I)

A solution of 1 g of 5-methyl-6-(pentafluorophenyl)-7-chloro-1,2,4-triazolo-[1,5a]pyrimidine (from example 2c) and 0.33 g of 4-methylpiperidine in 6 ml of butanol was stirred at 90° C. for 1 hour. After cooling to 20-25° C., the volatile components were distilled off. The residue was taken up in ethyl acetate, the organic phase was extracted with water and dried and the solvent was then removed. The residue gave, after chromatography on silica gel (cyclohexane/ethyl acetate mixtures), 0.9 g of the title compound 5-methyl-6-(pentafluorophenyl)-7-(4-methylpiperidinyl)-1,2,4-triazolo-[1,5a]pyrimidine together with the isomeric 7-methyl-6-(pentafluorophenyl)-5-(4-methylpiperidinyl)-1,2,4-triazolo[1,5a]pyrimidine in a ratio of 5:1 (according to ¹H-NMR).

¹H-NMR (CDCl₃, δ in ppm): 8.3 (s, 1H); 3.7 (d, broad, 2H); 2.8 (t, broad, 2H); 2.55 (s, 3H); 1.6 (m, 2H); 1.35 (m, 1H); 1.0 (m, 2H); 0.9 (d, 3H).

TABLE I
Compounds of the formula I

| | | | | phys. data |
|---|---|---|---|---|
| | | | | (¹H-NMR [δ ppm]; |
| No. | R¹ | R² | X | m.p. [° C.]) |
| I-1 | —(CH₂)₂CH(CH₃)(CH₂)₂— | | CN | 181-185 |
| I-2 | CH₂CH₃ | CH₂CH₃ | CN | 153-158 |
| I-3 | —(CH₂)₂CH(CH₃)(CH₂)₂— | | CH₃ | (cf. Example 2d) |
| I-4 | —CH₂—C(═CH₂)—CH | CH₂CH₃ | CH₃ | 8.25 (s); 2.5 (s); |
| | | | | 1.15 (t) |
| I-5 | —CH(CH₃)₂ | H | CH₃ | 8.2 (s); 2.5 (s); |
| | | | | 1.2 (d) |

Examples of the Action Against Harmful Fungi

The fungicidal action of the compounds of the formula I was demonstrated by the following experiments:

The active compounds were prepared as a stock solution with 0.25% by weight of active compound in acetone or DMSO. 1% by weight of the emulsifier Uniperol® EL (wetting agent having emulsifying and dispersing action based on ethoxylated alkylphenols) was added to this solution, and the mixture was diluted with water to the desired concentration.

### Use Example 1

### Activity Against Net Blotch of Barley Caused by Pyrenophora teres, Protective Application

Leaves of pot-grown barley seedlings of the cultivar “Hanna” were sprayed to run off point with an aqueous suspension having the concentration of active compound stated below. 24 hours after the spray coating had dried on, the test plants were inoculated with an aqueous suspension of Pyrenophora [syn. Drechslera] teres, the causative agent of net blotch. The test plants were subsequently placed in a greenhouse at 20-24° C. and 95-100% relative atmospheric humidity. After 6 days, the extent of development of the disease was determined visually in percent infection of the total leaf area.

In this test, the plants which had been treated with 250 ppm of the compound I-3 showed an infection of only 5%, whereas the untreated plants were 90% infected.

### Use Example 2

### Activity Against Gray Mold on Bell Pepper Leaves Caused by Botrytis cinerea, Protective Application

Bell pepper seedlings of the cultivar “Neusiedler Ideal Elite” were, after 2 to 3 leaves were well developed, sprayed to runoff point with an aqueous suspension having the concentration of active compound stated below. The next day, the treated plants were inoculated with a spore suspension of Botrytis cinerea which contained 1.7×10⁶ spores/ml in a 2% strength aqueous biomalt solution. The test plants were then placed in a dark climatized chamber at 22-24° C. and high atmospheric humidity. After 5 days, the extent of the fungal infection on the leaves could be determined visually in %.

In this test, the plants which had been treated with 63 ppm of the compounds I-1, I-3 or I-4 showed no infection, whereas the untreated plants were 90% infected.

### Use Example 3

### Protective Activity Against Septoria Leaf Spot of Wheat Caused by Septoria tritici

Leaves of potted wheat seedlings of the cultivar “Riband” were sprayed to runoff point with an aqueous suspension having the concentration of active compound stated below. 24 hours after the spray coating had dried on, they were inoculated with an aqueous spore suspension of Septoria tritici. The suspension contained 2.0×10⁶ spores/ml. The test plants were then placed in a greenhouse at temperatures between 18 and 22° C. and a relative atmospheric humidity close to 100%. After 2 weeks, the extent of the development of the disease was determined visually in % infection of the total leaf area.

In this test, the plants which had been treated with 63 ppm of the compounds I-1, I-2, I-3, I-4 or I-5 showed an infection of not more than 5%, whereas the untreated plants were 80% infected.

1. A 6-pentafluorophenyltriazolopyrimidine of the formula I 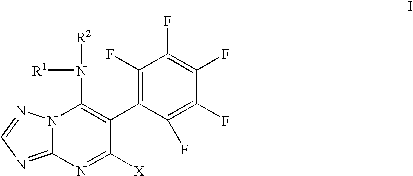
in which the substituents are as defined below:
R¹ is C₃-C₆-cycloalkyl, which can be substituted by C₁-C₄-alkyl, or is a group A: 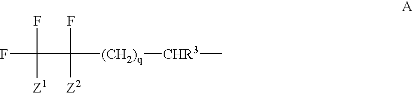
in which
Z¹ is hydrogen, fluorine or C₁-C₆-fluoroalkyl,
Z² is hydrogen or fluorine, or
Z¹ and Z² together form a double bond;
q is 0 or 1; and
R³ is hydrogen or methyl.
R² is hydrogen, methyl or ethyl;
R¹ and R² together with the nitrogen atom to which they are attached may also form a five- or six-membered heterocyclyl or heteroaryl which is attached via N and may contain a further heteroatom from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkyleneoxy;
or R¹ and R² together with the nitrogen atom to which they are attached may also form a group 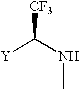
where Y is hydrogen or C₁-C₄-alkyl;
X is cyano, C₁-C₄-alkyl, C₁-C₄-alkoxy, C₃-C₄-alkenyloxy, C₁-C₂-haloalkoxy or C₃-C₄-haloalkenyloxy.

2. The compound of the formula I according to claim 1, in which X is cyano, C₁-C₄-alkoxy, C₃-C₄-alkenyloxy, C₁-C₂-haloalkoxy or C₃-C₄-haloalkenyloxy.

3. The compound of the formula I according to claim 1, in which X is cyano.

4. The compound of the formula I according to claim 1, in which X is methoxy.

5. The compound of the formula I according to claim 1, in which X is C₁-C₄-alkyl.

6. The compound of the formula I according to claim 1, in which R¹ and R² are as defined below:
R¹ is CH(CH₃)—CF₃, cyclopentyl or cyclohexyl;
R² is hydrogen or methyl;
R¹ and R² together form —(CH₂)₂CH(CH₃)(CH₂)₂—, —(CH₂)₂CH(CF₃)(CH₂)₂— or —(CH₂)₂O(CH₂)₂—.

7. A compound of the formula I.2. 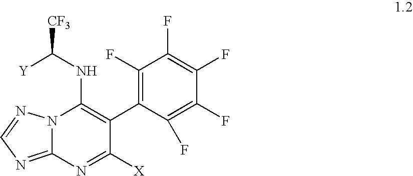
in which Y is hydrogen or C₁-C₄-alkyl and X is cyano, methyl, methoxy or ethoxy.

8. A compound of the formula I.3, 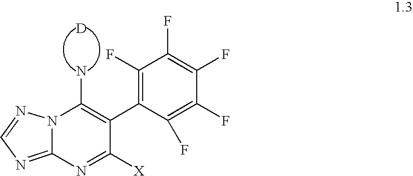
in which
D together with the nitrogen atom forms a five- or six-membered heterocyclyl or heteroaryl which is attached via N and may contain a further heteroatom from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy; and
X is cyano, methyl, methoxy or ethoxy.

9. A process for preparing the compounds of the formula I according to claim 2 by reacting 5-halo-6-(pentafluorophenyl)triazolopyrimidines of the formula II 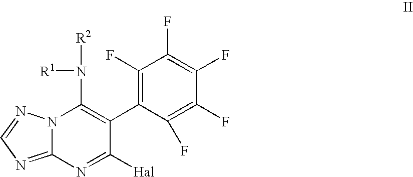
in which Hal is a halogen atom, with compounds of the formula III
M-X III
in which M is an ammonium, tetraalkylammonium or alkali metal or alkaline earth metal cation and X is as defined in claim 2.

10. A process for preparing compounds of the formula I according to claim 5 by reacting 2-aminotriazole of the formula IV 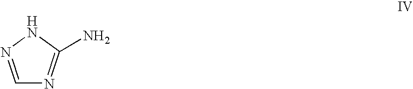
with keto esters of the formula V 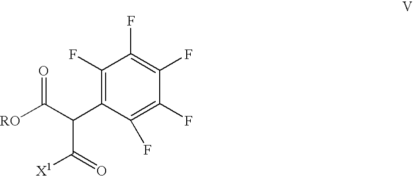
in which R and X¹ independently of one another are C₁-C₄-alkyl and L¹, L² and L³ are as defined in claim 1, to give 5-alkyl-7-hydroxy-6-phenyltriazolopyrimidines of the formula VI, 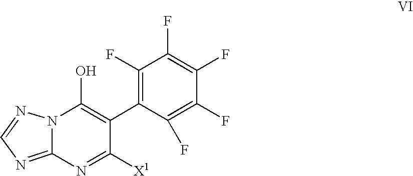
halogenation of VI with halogenating agents to give halopyrimidines of the formula VII 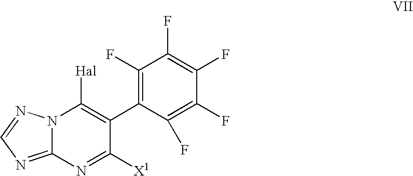
in which Hal is a halogen atom, and reaction of VII with amines of the formula VIII 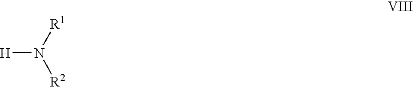
in which R¹ and R² are as defined in formula I.

11. A composition, comprising a solid or liquid carrier and a compound of the formula I according to claim 1.

12. Seed, comprising a compound of the formula I according to claim 1 in an amount of from 1 to 1000 g/100 kg.

13. A method for controlling phytopathogenic harmful fungi, which method comprises treating the fungi or the materials, plants, the soil or seeds to be protected against fungal attack with an effective amount of a compound of the formula I according to claim 1.

14. The compound of the formula I according to claim 2, in which X is cyano.

15. The compound of the formula I according to claim 2, in which X is methoxy.

16. The compound of the formula I according to claim 2, in which R¹ and R² are as defined below:
R¹ is CH(CH₃)—CF₃, cyclopentyl or cyclohexyl;
R² is hydrogen or methyl;
R¹ and R² together form —(CH₂)₂CH(CH₃)(CH₂)₂—, —(CH₂)₂CH(CF₃)(CH₂)₂— or —(CH₂)₂O(CH₂)₂—.

17. The compound of the formula I according to claim 3, in which R¹ and R² are as defined below:
R¹ is CH(CH₃)—CF₃, cyclopentyl or cyclohexyl;
R² is hydrogen or methyl;
R¹ and R² together form —(CH₂)₂CH(CH₃)(CH₂)₂—, —(CH₂)₂CH(CF₃)(CH₂)₂— or —(CH₂)₂O(CH₂)₂—.

18. The compound of the formula I according to claim 4, in which R¹ and R² are as defined below:
R¹ is CH(CH₃)—CF₃, cyclopentyl or cyclohexyl;
R² is hydrogen or methyl;
R¹ and R² together form —(CH₂)₂CH(CH₃)(CH₂)₂—, —(CH₂)₂CH(CF₃)(CH₂)₂— or —(CH₂)₂O(CH₂)₂—.

19. The compound of the formula I according to claim 5, in which R¹ and R² are as defined below:
R¹ is CH(CH₃)—CF₃, cyclopentyl or cyclohexyl;
R² is hydrogen or methyl;
R¹ and R² together form —(CH₂)₂CH(CH₃)(CH₂)₂—, —(CH₂)₂CH(CF₃)(CH₂)₂— or —(CH₂)₂O(CH₂)₂—.

20. A composition, comprising a solid or liquid carrier and a compound of the formula I according to claim 2.
